SEQUENCE LISTING (14 sequences)

SEQ ID NO: 1 - DNA, 20 bp, Artificial Sequence
  Description of Artificial Sequence Synthetic oligonucleotide
tatcgttcacggtcagcgac

SEQ ID NO: 2 - DNA, 20 bp, Artificial Sequence
  Description of Artificial Sequence Synthetic oligonucleotide
accctgatgggacgtacact

SEQ ID NO: 3 - DNA, 20 bp, Artificial Sequence
  Description of Artificial Sequence Synthetic oligonucleotide
tggggccactcgatccttga

SEQ ID NO: 4 - DNA, 20 bp, Artificial Sequence
  Description of Artificial Sequence Synthetic oligonucleotide
gacctgggtcacttgtgccg

SEQ ID NO: 5 - DNA, 20 bp, Artificial Sequence
  Description of Artificial Sequence Synthetic oligonucleotide
gcggccatggctccaagcaa

SEQ ID NO: 6 - DNA, 20 bp, Artificial Sequence
  Description of Artificial Sequence Synthetic oligonucleotide
cccaccgcacgttcagaagt

SEQ ID NO: 7 - DNA, 20 bp, Artificial Sequence
  Description of Artificial Sequence Synthetic oligonucleotide
cggcaagacgcggaagctca

SEQ ID NO: 8 - DNA, 20 bp, Artificial Sequence
  Description of Artificial Sequence Synthetic oligonucleotide
cccctaccatgactttattc

SEQ ID NO: 9 - DNA, 20 bp, Artificial Sequence
  Description of Artificial Sequence Synthetic oligonucleotide
acagatatggcaactcccag

SEQ ID NO: 10 - DNA, 20 bp, Artificial Sequence
  Description of Artificial Sequence Synthetic oligonucleotide
aatatgactagcaacaagtc

SEQ ID NO: 11 - DNA, 20 bp, Artificial Sequence
  Description of Artificial Sequence Synthetic oligonucleotide
gacggctgaggagcggaaga

SEQ ID NO: 12 - DNA, 20 bp, Artificial Sequence
  Description of Artificial Sequence Synthetic oligonucleotide
tgtggaggtgagcaatcccc

SEQ ID NO: 13 - DNA, 20 bp, Artificial Sequence
  Description of Artificial Sequence Synthetic oligonucleotide
acggccacgcagacttcatg

SEQ ID NO: 14 - DNA, 20 bp, Artificial Sequence
  Description of Artificial Sequence Synthetic oligonucleotide
ggacttctggttgtcgcaag

(19) United States
(12) Patent Application Publication
(10) Pub. No.: US 2022/0409663 A1
(43) Pub. Date: Dec. 29, 2022

(54) ENGINEERED T CELLS AND TUMOR-INFILTRATING LYMPHOCYTES TO OVERCOME IMMUNOSUPPRESSION IN THE TUMOR MICROENVIRONMENT

(71) Applicant: BOARD OF REGENTS, THE UNIVERSITY OF TEXAS SYSTEM, Austin, TX (US)

(72) Inventors: Samantha Marie FIX, Houston, TX (US); Katy REZVANI, Houston, TX (US); Patrick HWU, Houston, TX (US); Simone PUNT, Houston, TX (US); Soraya Zorro MANRIQUE, Houston, TX (US)

(21) Appl. No.: 17/756,074

(22) PCT Filed: Nov. 25, 2020
(86) PCT No.: PCT/US2020/062311

§ 371 Date: May 16, 2022

Related U.S. Application Data
(60) Provisional application No. 62/941,670, filed on Nov. 27, 2019.

(51) Int. Cl. A61K 35/17; C12N 15/90; A61K 45/06; A61K 38/17; A61P 35/00; C12N 15/113

(52) U.S. Cl. CPC A61K 35/17; C12N 15/907; A61K 45/06; A61K 38/1774; A61P 35/00; C12N 15/1136; C12N 15/1138; C12N 2310/14; C12N 2310/531

(57) ABSTRACT

Embodiments of the disclosure provide methods and compositions that facilitate cancer treatment including at least because they concern therapies that circumvent the tumor microenvironment. In specific embodiments, compositions are utilized for therapy that utilize tumor-infiltrating lymphocytes and/or engineered T cells that are protected from immunosuppression from the tumor microenvironment because they are engineered to have reduced or eliminated expression of transforming growth factor-beta receptor 2 and/or I-cell-Ig-and-ITIM-domain and/or CD7 genes.

This application claims priority to U.S. Provisional Patent Application Ser. No. 62/941,670, filed Nov. 27, 2019, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Nov. 12, 2020, is named UTSC_P1200WO_SL.txt and is 3,664 bytes in size.

### TECHNICAL FIELD

Embodiments of the disclosure concern at least the fields of cell biology, molecular biology, immunology, and medicine.

### BACKGROUND

Cellular immunotherapy holds much promise for the treatment of cancer. However, certain cellular therapies have limited success because of inhibitory signals from cancer cells or cells in the tumor microenvironment. For example, the induction of cells (e.g., regulatory T cells or myeloid-derived suppressor cells) in the microenvironment releases substances such as transforming growth factor-β (TGFβ) and adenosine that suppress the immune response and promote tumor cell proliferation and survival. Thus, there is an unmet need for improved methods of cellular immunotherapy.

### BRIEF SUMMARY

Embodiments of the disclosure encompass methods and compositions for adoptive cell therapy cancer treatment. The disclosure in particular provides methods and compositions to allow tumor-infiltrating lymphocytes (TILs) and engineered T cells to be more effective for cancer treatment than in the absence of the disclosed, respective methods and compositions. In specific embodiments, the disclosure provides methods and compositions to allow engineered TILs and/or engineered T cells to be more effective in tumor microenvironments compared to use of TILs and/or engineered T cells in the absence of the disclosed methods and compositions. In particular embodiments, the TILs and/or engineered T cells are autologous with respect to a recipient individual, although in some cases the TILs and/or engineered T cells are allogeneic with respect to a recipient individual.

The disclosure provides approaches to improvements on cancer immunotherapy, particularly with respect to use of engineered TILs and/or engineered T cells. In particular embodiments, knocking out one or more particular genes in TILs or T cells or knocking down one or more particular genes in TILs and/or T cells overcome immunosuppression in the tumor microenvironment. In specific embodiments, the gene(s) that are knocked out facilitate immunosuppression in the tumor microenvironment because they allow the engineered TILs or engineered T cells to circumvent one or more inhibitory signals in or from the tumor microenvironment. In specific embodiments, the engineered TILs and/or engineered T cells have reduced or eliminated expression of transforming growth factor-beta receptor 2 (TGFBR2) and/or T-cell-Ig-and-ITIM-domain (TIGIT) endogenous genes and/or CD7 and/or programmed cell death protein 1 (PD-1) and/or T-cell immunoglobulin and mucin-domain containing-3 (TIM-3). Although the engineered TILs and/or engineered T cells may be engineered using any suitable means to edit endogenous genes in the cells, in specific embodiments CRISPR is utilized.

In particular aspects of the disclosure, autologous TILs and/or T cells are used for an individual that has a tumor, although in alternative embodiments the individual has a hematological malignancy. In certain embodiments, TILs and/or T cells are obtained from the individual in need of cancer therapy, such as obtained from an individual's own cancer (tumor). The individual may be known to have cancer and have cells taken from the cancer for obtaining TILs, in some cases. In other cases, an individual may not be known to have cancer, and the TILs are obtained from the cancer upon being diagnosed for the cancer (for example, by biopsy). In any case, TILs may be taken from the cancer of an individual, expanded to a suitable number of expanded TILs, engineered to have knockout or knockdown of TGFBR2 and/or TIGIT and/or CD7 and/or PD-1 and/or TIM-3, and delivered back into the individual from which the TILs were originally obtained.

Embodiments of the disclosure include a composition, comprising: (a) engineered tumor-infiltrating lymphocytes (TILs), wherein said TILs comprise one or more of (1) disruption of expression and/or activity of transforming growth factor-beta receptor 2 (TGFBR2); (2) disruption of expression and/or activity of T-cell-Ig-and-ITIM-domain (TIGIT); (3) disruption of expression and/or activity of CD7; (4) disruption of expression of PD-1; and (5) disruption of expression of TIM-3, all of which are endogenous to the TILs; and/or

(b) engineered T cells, wherein said T cells comprise one or more of (1) disruption of expression and/or activity of transforming growth factor-beta receptor 2 (TGFBR2) endogenous to the TILs; (2) disruption of expression and/or activity of T-cell-Ig-and-ITIM-domain (TIGIT); and (3) disruption of expression and/or activity of CD7; (4) disruption of expression of PD-1; and (5) disruption of expression of TIM-3, all of which are endogenous to the T cells.

In specific embodiments the TILs are expanded TILs. The disruption of expression and/or activity of one or more of TGFBR2, TIGIT, CD7, PD-1, and TIM-3 may comprise nucleic acid, peptide, protein, small molecule, or a combination thereof. The nucleic acid may comprise siRNA, shRNA, anti-sense oligonucleotides, or guide RNA for CRISPR corresponding to TGFBR2, TIGIT, CD7, PD-1, or TIM-3, respectively. In specific cases, the TILs and/or the T cells comprise disruption of expression of TGFBR2, TIGIT, CD7, PD-1, and/or TIM-3. The T cells may comprise a heterologous antigen receptor that targets one or more cancer antigens, such as a T cell receptor, chimeric antigen receptor, chemokine receptor, chimeric cytokine receptor, or a mixture thereof.

In some embodiments, there is a population of cells of the composition of the disclosure, including wherein the population is in a pharmaceutically acceptable carrier.

In one embodiment, there is a method of preparing the cells encompassed herein, comprising the step of electroporating TILs and/or T cells, respectively with: (a) Cas9 or a nucleic acid that encodes Cas9; and one or more of (b), (c), (d): (b) a TGFBR2 guide RNA for CRISPR; (c) a TIGIT guide RNA for CRISPR, (d) a CD7 guide RNA for CRISPR, (e) a PD-1 guide RNA for CRISPR, or (f) a TIM-3 guide RNA for CRISPR. In specific embodiments, the method is further defined as comprising two or more electroporation steps, wherein a first electroporation step subjects the TILs and/or T cells to a one or more of TGFBR2 guide RNA, TIGIT guide RNA, CD7 guide RNA, PD-1 guide RNA, or TIM-3 guide RNA, and a second electroporation step subjects the TILs and/or T cells to guide RNAs for one or more of TGFBR2, TIGIT, CD7, PD-1 or TIM-3 that were not used in the first electroporation step. In an example wherein a third electroporation step is employed to target a third gene, the third electroporation step subjects the TILs and/or T cells to guide RNAs other than those used in the first and second electroporation steps, and so on, including for fourth and fifth electroporation steps when desired. The method may further comprise at least one step of expanding the TILs and/or T cells. In specific cases, there is an expansion step for the TILs and/or T cells prior to an electroporation step and/or there is an expansion step for the TILs and/or T cells after to an electroporation step.

In one embodiment, there is a method of killing cancer cells in an individual, comprising the step of delivering to the individual a therapeutically effective amount of the composition of the disclosure. The cancer may be a hematological cancer or comprises a solid tumor. In specific embodiments, the TILs and/or T cells are allogeneic or autologous with respect to the individual. The method may be further defined as (a) obtaining cancer cells from the individual; (b) expanding TILs from the cancer cells to produce expanded TILs; (c) engineering the expanded TILs to have (1) disruption of expression or activity of TGFBR2 endogenous to the TILs; and/or (2) disruption of expression or activity of TIGIT to produce engineered cells; and/or (3) disruption of expression or activity of CD7; and/or (4) disruption of expression of PD-1; and/or (5) disruption of expression of TIM-3; and (d) administering an effective amount of the engineered cells to the individual. The method may be further defined as (a) obtaining cancer cells from the individual; (b) expanding T cells to produce expanded T cells; (c) engineering the expanded T cells to have (1) disruption of expression or activity of TGFBR2 endogenous to the TILs; and/or (2) disruption of expression or activity of TIGIT to produce engineered cells; and/or (3) disruption of expression or activity of CD7; and/or (4) disruption of expression of PD-1; and/or (5) disruption of expression of TIM-3; and (d) administering an effective amount of the engineered cells to the individual.

In some cases, the individual is delivered an additional cancer therapy, such as surgery, radiation, chemotherapy, hormone therapy, immunotherapy, or a combination thereof.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1: Efficient delivery of Cas9 ribonucleoprotein (RNP) complexes into T cells for gene editing using electroporation-based transfection. This figure demonstrates that following electroporation nearly all cells are positive for the Cas9 RNP complex.

FIG. 2. Knockout of a model gene (Selplg) in murine T cells 5-days after electroporation-based delivery of the Cas9 RNP complex.

FIG. 3. Knockout of TIGIT in murine T cells using electroporation-based delivery of Cas9 RNP complexes. Data provided here shows TIGIT knockout at the RNA level as assessed by RT-PCR.

FIG. 4. Transfection efficiency in ex vivo-expanded patient-derived TIL. Transfer of TIGIT-specific RNP complexes by electroporation resulted in a cellular delivery efficiency of 76.5% (percent positive cells).

FIG. 5. Transfecting previously-expanded TIL with Cas9 RNP complexes targeting TIGIT results in appreciable knockout. The percentage of total live TIL positive for TIGIT in a control case (non-transfected) and an example case (transfected with TIGIT-specific RNP) are provided.

FIGS. 6A-6B. Identification of genes that regulate T cell infiltration into tumors through pooled shRNA screens in vivo. FIG. 6A. Schematic representation of the experimental design. Activated pmel T cells were transduced with a pooled shRNA library targeting 300 genes encoding proteins expressed on the cell surface, and cells were adoptively transferred (ACT) into irradiated B16 tumor-bearing mice. 7 days post ACT, pmel T cells were isolated from B16 tumors and spleens paired samples, and DNA isolation and sequencing were performed. FIG. 6B. Density plot. The arrow in the density plot indicates the enriched hairpins in the TIL population compared to splenic T cells and references (samples acquired before ACT). Analysis was done for 2-3 samples per group. Representative surface T cell screen is shown.

FIG. 7. Enhancement of T cell infiltration in tumor versus spleen through Cd7 knockdown based on shRNA barcodes. The number of shRNA barcode reads for each of the 10 different shRNA constructs targeting Cd7 in both the spleen and tumor samples (n=6 each) is shown. The majority of constructs show enrichment in the tumor samples compared to the spleen samples.

FIG. 8. Enrichment of Cd7 knockdown Pmel in tumors versus spleens based on individual gene knockdown. Pmel T cells were separately transduced with either a Cd7 shRNA containing lentiviral vector or a non-targeting control (NTC) vector, FACS-sorted for vector-expressed GFP and expanded before ACT into tumor-bearing mice. Total tumor infiltrating immune lymphocytes (TILs) were isolated from the tumors 12 days after ACT and counted. The Cd7 knockdown Pmel were found in higher numbers compared with untransduced or NTC-construct transduced Pmel T cells, confirming the effect found in the shRNA screen.

FIG. 9. Optimization of CRISPR gene knockout in patient-derived TIL using the T cell receptor alpha chain gene (TRAC) as a model target, using a variety of electroporation pulse parameters (denoted EH100, EN138, EH115, and E0115) and two different quantities of Cas9 input (5 g and 10 g).

FIG. 10. Optimization of CRISPR gene knockout of TIGIT in patient-derived TIL. TIL subjected to TIGIT knockout with various guide RNA sequences (denoted TIGIT AA, AB, AC, AD, and AE) show reduced TIGIT surface expression.

FIG. 11. Optimization of CRISPR gene knockout of TGFBR2 in patient-derived TIL. TIL were genetically modified using guide RNAs targeting TGFBR2 (different guide RNA sequences denoted with TGFBR2 AA, AB, AC, and AD).

FIG. 12. TIL that have undergone CRISPR gene knockout of TGFBR2 are resistant to the effects of exogenous TGF-β stimulation. TIL were genetically modified using guide RNAs targeting TGFBR2 (different guide RNA sequences denoted with TGFBR2 AA, AB, AC, and AD).

Table 1. Guide RNA sequences used to target TIGIT and TGFBR2 in human T cells.

FIG. 13. TIL that have undergone CRISPR gene knockout of TGFBR2 are resistant to the effects of exogenous TGF-β stimulation. Unmodified and modified TIL were cultured in the presence of TGF-β for 3 days. Data are presented as the fold change (ratio) of cytokine concentration from TGF-β-treated (10 ng/ml) to vehicle treated TIL. Data are presented from TIL isolated from two independent donors.

FIGS. 14A-14C. RNP transfection induces highly efficient Cas9/CRISPR-mediated PD-1 knockout in activated mouse CD8+ T cells. PD-1 protein expression evaluated by flow cytometry, six days after transfection; positive expression determined based on FMO (FIG. 14A). (FIG. 14B) In vitro activation with anti-CD3 and interleukin (IL)-2 upregulates cell-surface expression of PD-1 in CD8 T cells (control cells transfected with non-targeting control RNP, NTC condition). (FIG. 14C) T cells transfected with Cas9/gRNA RNP targeting PD-1, PD-1 KO condition, display a reduction of PD-1 expression.

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. In specific embodiments, aspects of the disclosure may “consist essentially of” or “consist of” one or more sequences of the disclosure, for example. Some embodiments of the invention may consist of or consist essentially of one or more elements, method steps, and/or methods of the disclosure. It is contemplated that any method or composition described herein can be implemented with respect to any other method or composition described herein. The scope of the present application is not intended to be limited to the particular embodiments of the process, machine, manufacture, composition of matter, means, methods and steps described in the specification. As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

The phrase “therapeutically effective amount” as used herein means that amount of a compound, material, or composition comprising a compound of the present disclosure that is effective for producing some desired therapeutic effect, e.g., treating (i.e., preventing and/or ameliorating) cancer in a subject, or inhibiting TGF-beta interactions with other molecules directly or indirectly, at a reasonable benefit/risk ratio applicable to any medical treatment. In one embodiment, the therapeutically effective amount is enough to reduce or eliminate at least one symptom. One of skill in the art recognizes that an amount may be considered therapeutically effective even if the cancer is not totally eradicated but improved partially. For example, the spread of the cancer may be halted or reduced or delayed in onset, a side effect from the cancer may be partially reduced or completely eliminated or delayed in onset, the life span of the subject may be increased, the subject may experience less pain, the quality of life of the subject may be improved, and so forth.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a “mammal” is an appropriate subject for the method of the present invention. A mammal may be any member of the higher vertebrate class Mammalia, including humans; characterized by live birth, body hair, and mammary glands in the female that secrete milk for feeding the young. Additionally, mammals are characterized by their ability to maintain a constant body temperature despite changing climatic conditions. Examples of mammals are humans, cats, dogs, cows, mice, rats, horses, goats, sheep, and chimpanzees. Mammals may be referred to as “patients” or “subjects” or “individuals”.

As used herein, a “disruption” of a gene refers to the elimination or reduction of expression of one or more gene products encoded by the subject gene in a cell, compared to the level of expression of the gene product in the absence of the disruption. Exemplary gene products include mRNA and protein products encoded by the gene. Disruption in some cases is transient or reversible and in other cases is permanent. Disruption in some cases is of a functional or full length protein or mRNA, despite the fact that a truncated or non-functional product may be produced. In some embodiments herein, gene activity or function, as opposed to expression, is disrupted. Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In some cases, the term is referring to cells that have been modified by the hand of man to harbor or express one or more molecules that are not found in nature.

With respect to heterologous antigen receptors, the term “engineered” as used herein refers to antigen receptors that are generated by the hand of man and are not found in nature and are not endogenous to the cell that expresses it. The receptors may be synthetically generated through standard recombination techniques, for example. The term includes the generation of fusion proteins comprising components that are not found together in nature, including in the same molecule. Examples include T cell receptors, chimeric antigen receptors, chimeric cytokine receptors, and so forth. The term “heterologous” as used herein refers to being derived from a different cell type or a different species than the recipient. In specific cases, it refers to a gene or protein that is synthetic and/or not from a T cell or TIL. The term also refers to synthetically derived genes or gene constructs. For example, a cytokine may be considered heterologous with respect to a T cell or TIL even if the cytokine is naturally produced by the T cell or TIL because it was synthetically derived, such as by genetic recombination, including provided to the T cell or TIL in a vector that harbors nucleic acid sequence that encodes the cytokine.

The present disclosure concerns improvements on cell therapies for an individual having cancer and in need of cancer treatment. The cells are non-natural and engineered by the hand of man to have one or more gene modifications to endogenous gene(s) of the cells. In particular embodiments, the cells are tumor-infiltrating lymphocytes (TILs) and/or T cells. In particular cases, there is the knocking out or knocking down of genes that encode inhibitory receptors on TILs cells and/or T cells to improve the therapeutic function of these cells, including in the context of adoptive cell therapy. The disclosure provides improvements on TIL therapy and/or T cell therapy in specific aspects, such as where the cells are isolated from a patient's tumor, expanded to a suitable number, and reinfused into the same patient. In specific embodiments, knockout of TGFBR2 and/or TIGIT and/or CD7 and/or PD-1 and/or TIM-3 allows these cells to overcome key immunosuppressive signals in the tumor microenvironment. In at least certain cases, knockout is performed by transfecting TIL cells and/or T cells with Cas9 ribonucleoprotein (RNP) complexes comprised of the Cas9 protein and gRNA targeting each gene of interest. As encompassed herein, there is efficient knockout of genes in human and mouse T cells and TILs and/or T cells using this methodology.

### I. Engineered Tumor-Infiltrating Lymphocytes

Embodiments of the disclosure provide for one or more cellular compositions for treatment of any cancer. The cellular compositions may comprise genetically modified TILs (such as having reduction in expression of one or more endogenous genes produced by the hand of man, as opposed to natural mutations) and include formulations for administration to an individual in need of cancer treatment. The compositions may or may not be formulated for storage, transport, and/or delivery.

Embodiments of the disclosure include cells for immunotherapy that include TILs that are engineered to be more effective at cancer treatment than TILs that are not so engineered. In some embodiments, the TILs are engineered to have (1) reduction or elimination of expression of endogenous TGF-beta Receptor 2 (TGFBR2) and/or reduction or elimination of activity of the expressed protein; and/or (2) reduction or elimination of expression of endogenous T-cell-Ig-and-ITIM-domain (TIGIT) and/or reduction or elimination of activity of the expressed protein; and/or (3) reduction or elimination of expression of endogenous CD7 and/or reduction or elimination of activity of the expressed protein; and/or (4) reduction or elimination of expression of endogenous PD-1 and/or reduction or elimination of activity of the expressed protein; and/or (5) reduction or elimination of expression of endogenous TIM-3 and/or reduction or elimination of activity of the expressed protein. Such engineering may occur by any suitable means. Thus, the TILs may be gene edited, and the gene editing may occur by any means. The gene editing may or may not be transient; in specific cases the gene editing is permanent.

In some embodiments, the gene disruption is carried out by effecting a disruption in one or more of the desired genes, such as a knock-out, insertion, missense or frameshift mutation, including biallelic frameshift mutation, deletion of all or part of the gene, e.g., one or more exon or portion therefore, and/or knock-in, as some examples. In certain cases, the disruption can be affected be sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the TGFBR2 gene or a portion thereof or the TIGIT gene or a portion thereof or the CD7 gene or a portion thereof or the PD-1 gene or a portion thereof or the TIM-3 gene or a portion thereof.

In some embodiments, TGFBR2 gene disruption and/or TIGIT gene disruption and/or CD7 gene disruption and/or PD-1 gene disruption and/or TIM-3 gene disruption is performed by induction of one or more double-stranded breaks and/or one or more single-stranded breaks in the gene, including in a targeted manner. In some embodiments, the double-stranded or single-stranded breaks are made by a nuclease, e.g., an endonuclease, such as a gene-targeted nuclease. In some aspects, the breaks are induced in the coding region of the gene, e.g., in an exon. For example, in some embodiments, the induction occurs near the N-terminal portion of the coding region, e.g., in the first exon, in the second exon, or in a subsequent exon.

In some embodiments, gene disruption is achieved using antisense techniques, including by RNA interference (RNAi), short interfering RNA (siRNA), short hairpin (shRNA), and/or ribozymes are used to selectively suppress or repress expression of the gene. siRNA technology is RNAi that employs a double-stranded RNA molecule having a sequence homologous with the nucleotide sequence of mRNA that is transcribed from the gene, and a sequence complementary with the nucleotide sequence. siRNA generally is homologous/complementary with one region of mRNA that is transcribed from the gene, or may be siRNA including a plurality of RNA molecules that are homologous/complementary with different regions. In some aspects, the siRNA is comprised in a polycistronic construct.

For disruption using techniques that utilize sequence knowledge of the target gene or gene product, an example of a TIGIT nucleic acid sequence is at GenBank® Accession No. EU675310 and its corresponding protein is at GenBank® Accession No. ACD74757. One example of a TGFBR2 nucleic acid sequence is at GenBank® Accession No. NM_001024847 and an example of its corresponding protein sequence is at GenBank® Accession No. NP_001020018. One example of a CD7 nucleic acid sequence is at GenBank® Accession No. NM_006137, and an example of its corresponding protein sequence is at GenBank® Accession No. NP_006128. One example of a PD-1 nucleic acid sequence is at GenBank® Accession No. L27440, and an example of its corresponding protein sequence is at GenBank® Accession No. AAC41700.1. One example of a TIM-3 nucleic acid sequence is at GenBank® Accession No. JX049979, and an example of its corresponding protein sequence is at GenBank® Accession No. AFO66593.1.

In some embodiments, the disruption is achieved using a DNA-targeting molecule, such as a DNA-binding protein or DNA-binding nucleic acid, or complex, compound, or composition, containing the same, which specifically binds to or hybridizes to the TGFBR2 gene or the TIGIT gene or the CD7 gene or the PD-1 gene or the TIM-3 gene, respectively. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease. Zinc finger, TALE, and CRISPR system binding domains can be engineered to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data.

In cases where gene alteration is carried out by induction of one or more double-stranded breaks and/or one or more single-stranded breaks in the gene, the double-stranded or single-stranded breaks may undergo repair via a cellular repair process, such as by non-homologous end-joining (NHEJ) or homology-directed repair (HDR). In some aspects, the repair process is error-prone and results in disruption of the gene, such as a frameshift mutation, e.g., biallelic frameshift mutation, which can result in complete knockout of the gene. For example, in some aspects, the disruption comprises inducing a deletion, mutation, and/or insertion. In some embodiments, the disruption results in the presence of an early stop codon. In some aspects, the presence of an insertion, deletion, translocation, frameshift mutation, and/or a premature stop codon results in disruption of the expression, activity, and/or function of the gene.

In some embodiments, the alteration is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

The TILs may be introduced to a guide RNA and CRISPR enzyme, or mRNA encoding the CRISPR enzyme. For CRISPR-mediated disruption, the guide RNA and endonuclease may be introduced to the TILs by any means known in the art to allow delivery inside cells or subcellular compartments of agents/chemicals and molecules (proteins and nucleic acids) can be used including liposomal delivery means, polymeric carriers, chemical carriers, lipoplexes, polyplexes, dendrimers, nanoparticles, emulsion, natural endocytosis or phagocytose pathway as non-limiting examples, as well as physical methods such as electroporation. In specific aspects, electroporation is used to introduce the guide RNA and endonuclease, or nucleic acid encoding the endonuclease.

In one exemplary method, the method for CRISPR knockout of multiple genes may comprise isolation of TILs from a cancer of the individual, including from a tumor. When obtained from an individual for autologous purposes, the TILs may be obtained by any suitable method such as through biopsy or routine sample collection of any kind, including from blood, bone marrow, and so forth. In cases wherein the TILs are allogeneic with respect to a recipient individual, the source of the TILs may be from storage, from a commercial source, fresh from a donor, and so forth.

In embodiments where the TILs are expanded, they may be expanded by any suitable method, such as initial expansion of TILs from tumor fragments via culture in IL-2 followed by a rapid expansion protocol involving stimulation via CD3 crosslinking and IL-2 with or without additional co-stimulation through 4-1BB/CD137 in the presence of peripheral blood mononuclear cells (PBMCs) or artificial antigen presenting cells.

Prior to or following expansion, the TILs may be subject to engineering to effect knockdown or knockout of TIGIT and/or TGFBR2 and/or CD7 and/or PD-1 and/or TIM-3. In cases wherein CRISPR is utilized, the engineering of TIGIT and/or TGFBR2 and/or CD7 and/or PD-1 and/or TIM-3 may occur in the same electroporation step or in successive electroporation steps. When the electroporation steps are successive, the knockout/knockdown of one or more of TIGIT, TGFBR2, CD7, PD-1 and TIM-3 may be before or after the knockout/knockdown, respectively, of one of TIGIT, TGFBR2, CD7, PD-1 and TIM-3 that was not already knocked down or out. Any combination of knockout/knockdown of TIGIT, TGFBR2, CD7, PD-1 and TIM-3 may occur in any order, for example where each of the desired genes are edited in a combination 2, 3, 4, or 5 electroporation steps. As one specific example only, TIGIT and TGFBR2 may be edited in a first electroporation step, and CD7 may be edited in a second or subsequent electroporation step (and any combination thereof, including for PD-1 and TIM-3). Following CRISPR editing of the TILs, they may or may not be subjected to an additional expansion step, for example through re-stimulation via CD3 crosslinking and IL2 stimulation.

In some aspects, a Cas nuclease and gRNA (including a fusion of crRNA specific for the target sequence and fixed tracrRNA) are introduced into the TIL. In general, target sites at the 5′ end of the gRNA target the Cas nuclease to the target site, e.g., the TGFBR2 gene or the TIGIT gene or the CD7 gene or the PD-1 gene or the TIM-3 gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). One can utilize a CRISPR enzyme that is mutated to have reduced off-targeting editing, such as from Integrated DNA Technologies, Inc. In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5- transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

In some embodiments, the alteration of the expression, activity, and/or function of the TGFBR2 and/or the TIGIT gene and/or the CD7 gene and/or the PD-1 gene and/or the TIM-3 gene is carried out by disrupting the corresponding gene. In some aspects, the gene is modified so that its expression is reduced by at least at or about 20, 30, or 40%, generally at least at or about 50, 60, 70, 80, 90, 91, 92, 93, 94, 95, 96, 97, 98, 99, or 100% as compared to the expression in the absence of the gene modification or in the absence of the components introduced to effect the modification.

In particular embodiments, in addition to the reduction in expression of one or more of TGFBR2, TIGIT, CD7, PD-1, and TIM-3, the TILs may be further modified by the hand of man.

In some embodiments, the TILs prior to or during the processes for generating the engineered TILs are subject to enrichment by negative or positive selection for one or more markers.

### II. Engineered T Cells

Embodiments of the disclosure provide for one or more T cell compositions for treatment of any cancer. The cellular compositions may comprise genetically modified T cells (such as having reduction in expression of one or more endogenous genes produced by the hand of man, as opposed to natural mutations) and include formulations for administration to an individual in need of cancer treatment. The compositions may or may not be formulated for storage, transport, and/or delivery.

In certain embodiments, the T cells are modified to have reduced or no expression of one or more endogenous genes. In particular embodiments, the T cells are engineered to express one or more heterologous antigen receptors, such as engineered TCRs, CARs, chimeric cytokine receptors, chemokine receptors, a combination thereof, and so on. The heterologous antigen receptors are synthetically generated by the hand of man. In particular embodiments, the T cells are modified to express a CAR and/or TCR having antigenic specificity for one or more cancer antigens. Multiple CARs and/or TCRs, such as to different antigens, may be added to the T cells. In some aspects, the T cells are engineered to express the CAR or TCR by knock-in of the CAR or TCR at a particular gene locus, such as by using CRISPR. In some embodiments, the T cells are engineered to have reduction in expression of one or more endogenous genes and are engineered to express one or more heterologous antigen receptors.

In some embodiments, the T cells are derived from the blood, bone marrow, lymph, umbilical cord, and/or lymphoid organs. In some aspects, the cells are human cells. The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the methods include isolating cells from a subject, preparing, processing, culturing, engineering them to express a synthetic antigen receptor (such as a non-native TCR), engineering them to have reduced or eliminated expression of TGRBR2, TIGIT, CD7, PD-1, and/or TIM-3, and re-introducing them into the same and/or different subject, before or after cryopreservation. Such steps may or may not occur in that particular order. For example, T cells may be engineered to have gene editing of the endogenous gene(s) following by engineering the gene edited cells to express a synthetic antigen receptor.

In particular embodiments, certain CRISPR nucleic acid reagents may be utilized in the T cells, including as follows (and also see Table 1):

TGFBR2 (Exon 5)
(SEQ ID NO: 11)
GACGGCTGAGGAGCGGAAGA
(gRNA1)
(SEQ ID NO: 12)
TGTGGAGGTGAGCAATCCCC
(gRNA2)

Examples of Mouse Sequences:

Mm.Cas9.TGFBR2.1.AA:
(SEQ ID NO: 13)
ACGGCCACGCAGACTTCATG
Mm.Cas9.TGFBR2.1.AB:
(SEQ ID NO: 14)
GGACTTCTGGTTGTCGCAAG

Among the sub-types and subpopulations of T cells (e.g., CD4+ and/or CD8+ T cells) are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, one or more of the engineered T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ T cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations.

In some embodiments, the engineered T cells are autologous T cells. In this method, tumor samples are obtained from individuals in need of cancer treatment, and a single cell suspension may or may not be obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests may be cultured with one or more specific interleukins, such as IL-2.

The cultured T cells can be pooled and rapidly expanded. Rapid expansion provides an increase in the number of antigen-specific T-cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days.

Expansion can be accomplished by any of a number of methods as are known in the art. For example, T cells can be rapidly expanded using non-specific T-cell receptor stimulation in the presence of feeder lymphocytes and either IL-2 or IL-15. The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as an human leukocyte antigen A2 (HLA-A2) binding peptide, in the presence of a T-cell growth factor, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2+ allogeneic lymphocytes and IL-2, for example.

In addition to having one or more heterologous antigen receptors and reduced expression of one or more of TGFBR2, TIGIT, CD7, PD-1, and TIM-3, the T cells may be modified to express one or more T cell growth factors that promote the growth and activation of the T cells. Suitable T cell growth factors include, for example, IL-2, IL-7, IL-15, and IL-12. Suitable methods of modification are known in the art. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994. In particular aspects, modified autologous T cells express the T cell growth factor at high levels. T cell growth factor coding sequences, such as that of IL-12, are readily available in the art, as are promoters, the operable linkage of which to a T cell growth factor coding sequence promote high-level expression.

A. T Cell Receptors

In some embodiments, the engineered heterologous antigen receptors include recombinant TCRs and/or TCRs cloned from naturally occurring T cells. A “T cell receptor” or “TCR” refers to a molecule that contains a variable α and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or Ca, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

B. Chimeric Antigen Receptors (CARs)

In some embodiments, the T cells are engineered to express one or more CARs comprising one or more extracellular antigen-recognition domains that specifically bind to an antigen. In some embodiments, the antigen is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

In some embodiments, the CAR comprises: a) one or more intracellular signaling domains, b) a transmembrane domain, and c) an extracellular domain comprising one or more antigen binding regions, which in specific embodiments is an scFv that binds the antigen.

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 A1.

In some embodiments, the CARs include activating or stimulatory CARs, costimulatory CARs (see WO2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acids, including nucleic acids encoding an antigen-specific CAR polypeptide, including in some cases a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be human genes used to enhance cellular immunotherapy for human patients. In a specific embodiment, the disclosure includes a full-length CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, DAP12, and 4-1BB (CD137). In addition to a primary signal initiated by CD3zeta, an additional signal provided by a human costimulatory receptor inserted in a human CAR is important for full activation of T cells and could help improve in vivo persistence and the therapeutic success of the adoptive immunotherapy.

In some embodiments, CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In certain embodiments of the chimeric antigen receptor, the antigen-specific portion of the receptor (which may be referred to as an extracellular domain comprising an antigen binding region) comprises a tumor associated antigen or a pathogen-specific antigen binding domain. Antigens include carbohydrate antigens recognized by pattern-recognition receptors, such as Dectin-1. A tumor associated antigen may be of any kind so long as it is expressed on the cell surface of tumor cells. Exemplary embodiments of tumor associated antigens include CD19, CD20, carcinoembryonic antigen, alphafetoprotein, CA-125, MUC-1, CD56, EGFR, c-Met, AKT, Her2, Her3, epithelial tumor antigen, melanoma-associated antigen, mutated p53, mutated ras, and so forth. In certain embodiments, the CAR may be co-expressed with one or more cytokines to improve persistence, for example when there is a low amount of tumor-associated antigen. For example, a CAR may be co-expressed with one or more cytokines, such as IL-7, IL-2, IL-15, IL-12, IL-18, IL-21, or a combination thereof.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In certain embodiments, the platform technologies disclosed herein to genetically modify T cells, comprise (i) non-viral gene transfer using an electroporation device (e.g., a nucleofector), (ii) CARs that signal through endodomains (e.g., CD28/CD3-ζ, CD137/CD3-ζ, or other combinations), (iii) CARs with variable lengths of extracellular domains connecting the antigen-recognition domain to the cell surface, and, in some cases, and (iv) artificial antigen presenting cells (aAPC) derived from K562 to be able to robustly and numerically expand CAR⁺ immune cells (Singh et al., 2008; Singh et al., 2011).

C. Antigens

Among the antigens targeted by the genetically engineered heterologous antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may be targeted in the present method. The antigen may be associated with certain cancer cells but not associated with non-cancerous cells, in some cases. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include CD19, EBNA, CD123, HER2, CA-125, TRAIL/DR4, CD20, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notchl-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAGiB, SUNC1, and LRRN1.

The sequences for antigens are known in the art, for example, in the GenBank® database, and including the following examples: CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRvIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, liver, brain, bone, stomach, spleen, testicular, cervical, anal, gall bladder, thyroid, or melanoma cancers, as examples. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

In other embodiments, an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, Staphylococcus species including Methicillin-resistant Staphylococcus aureus (MRSA), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gp120, gp41, p17, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (H1, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), IE1 and 1E2 (UL123 and UL122), protein products from the cluster of genes from UL128-UL150 (Rykman, et al., 2006), envelope glycoprotein B (gB), gH, gN, and pp150. As would be understood by the skilled person, CMV proteins for use as antigens described herein may be identified in public databases such as GenBank®, SWISS-PROT®, and TREMBL® (see e.g., Bennekov et al., 2004; Loewendorf et al., 2010; Marschall et al., 2009).

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al., 2008).

Antigens derived from respiratory syncytial virus (RSV) that are contemplated for use herein include any of the eleven proteins encoded by the RSV genome, or antigenic fragments thereof: NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use include any one of the five major proteins encoded by the VSV genome, and antigenic fragments thereof: large protein (L), glycoprotein (G), nucleoprotein (N), phosphoprotein (P), and matrix protein (M) (see, e.g., Rieder et al., 1999).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be bacterial antigens. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., 2007). As would be understood by the skilled person, Staphylococcus proteins for use as antigens may also be identified in other public databases such as GenBank®, Swiss-Prot®, and TrEMBL®.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneumoniae are also known in the art and may be used as an antigen in some embodiments (see, e.g., Zysk et al., 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®. Proteins of particular interest for antigens according to the present disclosure include virulence factors and proteins predicted to be exposed at the surface of the pneumococci (see, e.g., Frolet et al., 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g., S. pyogenes M proteins), group B streptococcus (S. agalactiae) polypeptides, Treponema polypeptides, and Yersinia polypeptides (e.g., Y pestis F1 and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

### III. Methods of the Disclosure

Embodiments of the disclosure include improved immunotherapy methods of treating or preventing any kind of medical condition, including at least cancer, including hematological malignancies or solid tumors, by using TILs and/or the T cells as at least part of the therapy. Hematological malignancies include at least cancers of the bone marrow, T or B cell malignancies, leukemias, lymphomas, blastomas, myelomas, and the like. Specific examples include at least Acute myeloid leukemia, B-cell acute lymphoblastic leukemia, T-cell acute lymphoblastic leukemia, Myelodysplastic syndromes, Chronic lymphocytic leukemia/small lymphocytic lymphoma, Follicular lymphoma, Lymphoplasmacytic lymphoma, Diffuse large B-cell lymphoma, Mantle cell lymphoma, Hairy cell leukemia, Plasma cell myeloma or multiple myeloma, Mature T/NK neoplasms, and so forth. Examples of solid tumors include tumors of the brain, lung, breast, prostate, pancreas, stomach, anus, head and neck, bone, skin, liver, kidney, thyroid, testes, ovary, endometrium, gall bladder, peritoneum, cervix, colon, rectum, vulva, spleen, a combination thereof, and so forth.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Methods of the disclosure encompass immunotherapies including adoptive cellular therapy, with TILs (whether expanded or not) and/or T cells (whether expanded or not) for treating cancer, where the immunotherapies are improved to allow greater efficacy for the immunotherapy by inhibiting released inhibitory TGF-beta (such as from cancer cells) or inhibiting associated interactions, such as the relationship between TGF-beta and integrins, or inhibiting the ability of TGF-beta to bind to the immune cells (by knocking out its receptor in the cells). Such modification of TILs and/or T cells allows for greater efficacy in cancer treatment.

In some embodiments, the present disclosure provides methods for immunotherapy comprising administering an effective amount of TILs and/or T cells of the present disclosure, wherein the TILs and/or T cells are particularly modified. In one embodiment, a medical disease or disorder is treated at least by particular TILs and/or T cells that elicit an immune response in the recipient. In certain embodiments of the present disclosure, any cancer is treated by transfer of a specific TIL and/or T cell population that elicits an immune response. Provided herein are methods for treating or delaying progression of cancer in an individual comprising administering to the individual an effective amount an antigen-specific cell therapy when the TILs and/or T cells comprise molecules, such as heterologous antigen receptors, that can target a desired antigen.

In particular embodiments of the present disclosure, an effective amount of TILs and/or T cells are delivered to an individual in need thereof, such as an individual that has cancer of any kind. The cells then enhance the individual's immune system to attack the cancer cells. In some cases, the individual is provided with one or more doses of the TILs and/or engineered T cells. In cases where the individual is provided with two or more doses of the TILs and/or T cells, the duration between the administrations should be sufficient to allow time for propagation in the individual, and in specific embodiments the duration between doses may be 1, 2, 3, 4, 5, 6, 7, or more days. Successive doses may or may not be identical in amount to one another. In some cases, the successive doses decrease over time or increase over time.

Methods of the disclosure encompass delivery of an effective amount of compositions comprising TILs and/or T cells engineered for knockout of TGFBR2 and/or TIGIT and/or CD7 and/or PD-1 and/or TIM-3. In some cases, there is a population of multiple cells, each of which single TILs and/or single T cells have knockout of TGFBR2 and TIGIT and CD7 and PD-1 and TIM-3, whereas in other cases the population is a mixture of TIL cells and/or T cells having TGFBR2 knocked out and/or TIGIT knocked out and/or CD7 knocked out and/or PD-1 knocked out and/or TIM-3 knocked out. In cases wherein an order of delivery of the two or more components is desired, the order may be of any kind so long as the delivery is therapeutically effective. In specific embodiments, delivery of TIL cells and/or T cells comprising knockout of the one or more desired genes occurs prior to a second therapy so that the second therapy becomes more effective than without the initial TIL and/or engineered T cell step.

In embodiments wherein both engineered TILs and engineered T cells are administered to an individual in need thereof, the engineered TILs and engineered T cells may or may not be in the same formulation. In cases wherein the engineered TILs and engineered T cells are in the same formulation, they may or may not be substantially equal in amount. For example, there may be a particular ratio utilized for the engineered TILs and engineered T cells. In specific cases, the ration may be 1:1, 1:2, 1:5, 1:10, 1:25, 1:50, 1:100, 1:250, 1:1000, 1:10000, and any ratio derivable there between. When the engineered TILs and engineered T cells are not in the same formulation, they may or may not be administered to the individual at the same time. Regardless of whether or not they are administered to the individual at the same time, they may or may not be delivered by the same route of administration. In specific cases, the engineered TILs and engineered T cells are administered intravenously, including in the same formulation. When they are administered separately, it may be in any order. For separate administrations, the duration of time between the administrations may be of any suitable duration in time, such as within 1-60 seconds, within 1-60 minutes, within 1-7 days, within 1-4 weeks, within 1-12 months, or longer, and any duration derivable there between.

In some embodiments in which both engineered TILs and engineered T cells are administered to an individual, their collective affect may be additive or synergistic with respect to treatment of the cancer in the individual.

### IV. Pharmaceutical Compositions

Pharmaceutical compositions of the present disclosure comprise an effective amount of engineered TILs and/or engineered T cells having modified expression of TGFBR2 and/or TIGIT and/or CD7 and/or PD-1 and/or TIM-3 (and/or reagents to generate same ex vivo or in vivo) dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises engineered TILs and/or engineered T cells (and/or reagents to generate same ex vivo or in vivo) will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The pharmaceutical compositions may comprise different types of carriers depending on whether it is to be administered in solid, liquid or aerosol form, and whether it need to be sterile for such routes of administration as injection. The presently disclosed compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art.

The engineered TILs and/or engineered T cells (and/or reagents to generate same ex vivo or in vivo) may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the compositions of the present disclosure suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include the engineered TILs and/or engineered T cells (and/or reagents to generate same ex vivo or in vivo), and optionally an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the engineered TILs and/or engineered T cells (and/or reagents to generate same ex vivo or in vivo) may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, one may utilize in a dose of cells 8-150×10⁹ cells. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In some embodiments of the present disclosure, the engineered TILs and/or engineered T cells (and/or reagents to generate same ex vivo or in vivo) are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations that are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, compositions may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,613,308; 5,466,468; 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound engineered TILs and/or engineered T cells (and/or reagents to generate same ex vivo or in vivo) may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present disclosure may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### V. Combination Therapies

In certain embodiments, the compositions and methods of the present embodiments involve a cancer therapy that is additional to the compositions comprising engineered TILs and/or engineered T cells. The additional therapy may be radiation therapy, surgery (e.g., lumpectomy and a mastectomy), chemotherapy, gene therapy, DNA therapy, viral therapy, RNA therapy, immunotherapy (other than that of the present disclosure), bone marrow transplantation, nanotherapy, monoclonal antibody therapy, hormone therapy, or a combination of the foregoing. The additional therapy may be in the form of adjuvant or neoadjuvant therapy.

In some embodiments, the additional therapy is the administration of one or more small molecule enzymatic inhibitors and/or one or more anti-metastatic agents. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent(s). The additional therapy may be one or more of the chemotherapeutic agents known in the art.

An immune cell therapy (in addition to the TIL therapy and/or engineered T cell therapy of the disclosure) may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from the composition(s) of the disclosure, one would generally ensure that a significant period of time did not expire between the time of each delivery, such that the two compounds would still be able to exert an advantageously combined effect on the patient. In such instances, it is contemplated that one may provide a patient with the immunotherapy therapy and the disclosed compositions within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Administration of any compound or cell therapy of the present embodiments to a patient will follow general protocols for the administration of such compounds, taking into account the toxicity, if any, of the agents. Therefore, in some embodiments there is a step of monitoring toxicity that is attributable to combination therapy.

A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

B. Radiotherapy

Other factors that cause DNA damage and have been used extensively include what are commonly known as 7-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

C. Immunotherapy

The skilled artisan will understand that additional immunotherapies (outside of the disclosed engineered TIL cell therapy and/or engineered T cell therapy) may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells other than those having knockdown or knockout of TGFBR2 and/or TIGIT.

Antibody-drug conjugates have emerged as a breakthrough approach to the development of cancer therapeutics. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons of any kind, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VI. Kits of the Disclosure

Any of the compositions described herein may be comprised in a kit. In a non-limiting example, TILs and/or T cells (and/or reagents to generate engineered of same), and these may be comprised in suitable container means in a kit of the present disclosure.

An article of manufacture or a kit is provided comprising engineered TILs and/or engineered T cells, and/or one or more reagents for generating them. The TILs and/or T cells may be from any source, and in specific embodiments the TILs and/or T cells have been produced by methods encompassed herein. In specific embodiments, the TILs and/or T cells have been gene edited and may be provided in the kit so that they may be further modified to express one or more heterologous antigen receptors. In specific embodiments, the TILs and/or T cells have been modified to express one or more heterologous antigen receptors and may be provided in the kit so that they may be further modified to be gene edited. In specific embodiments, one or more reagents for generating the TILs and/or T cells are provided in the kit, such as reagents that target a specific gene, reagents that comprise a heterologous antigen receptor (or one or more reagents to produce the heterologous antigen receptor), or a combination thereof. In general embodiments, the reagents may comprise nucleic acid including DNA or RNA, protein, media, buffers, salts, co-factors, and so forth. In specific cases, the kit comprises one or more CRISPR-associated reagents, including for targeting a specific desired gene.

The compositions of the kits may be packaged either in aqueous media or in lyophilized form. The container means of the kits will generally include at least one vial, test tube, flask, bottle, syringe or other container means, into which one or more components may be placed, and preferably, suitably aliquoted. Where there are more than one component in the kit, the kit also may generally contain a second, third or other additional container into which the additional components may be separately placed. However, various combinations of components may be comprised in a vial. The kits of the present disclosure also will typically include a means for containing the engineered TILs and/or engineered T cells (and/or reagents to generate same), and any other reagent containers, in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly envisioned. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the disclosure may also comprise, and/or be packaged with, an instrument for assisting with the injection/administration and/or placement of the ultimate composition within the body of an animal. Such an instrument may be a syringe, pipette, forceps, and/or any such medically approved delivery vehicle. In some embodiments, reagents or apparatuses or containers are included in the kit for ex vivo use.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Knocking Out Genes in Tumor-Infiltrating Lymphocytes to Overcome Immunosuppression in the Tumor Microenvironment

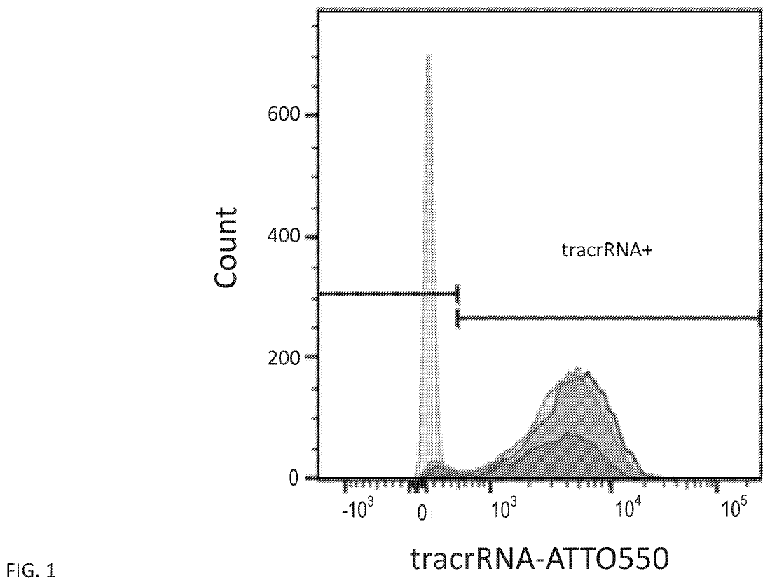

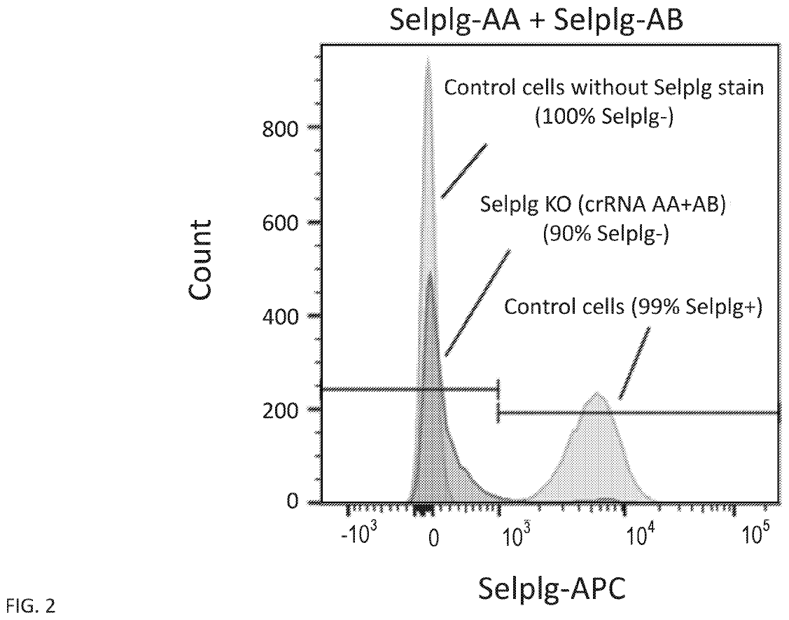

The present example demonstrates gene editing in TILs to provide them with enhanced activity in the tumor microenvironment. In specific methods, the TILs are knocked out for one or more specific endogenous genes in the TILs. The example provides a demonstration that delivering Cas9 protein that has been complexed with specific gRNA species can be used to efficiently perform CRISPR gene editing in immune cells, including murine T cells and human TIL. This approach is used to generate the TIL with reduced or eliminated expression of TGFBR2 and/or TIGIT and/or CD7. As an initial study, FIG. 1 shows efficient delivery of Cas9 RNP complexes (comprised of Alt-R S.p. Cas9 Nuclease V3, Alt-R® CRISPR-Cas9 tracrRNA—ATTO™ 550, and two distinct crRNAs specific for the Selplg gene (named AA and AB)) into T cells for gene editing using electroporation-based transfection, as one example, wherein Alt-R® CRISPR-Cas9 tracrRNA-ATTO™ 550 was utilized for fluorescent tracking of transfected cells. The achieved transfection efficiency was >97% in mouse CD8+ T cells using this technique (that is, >97% of cells are positive for the RNP complex 24 hrs post-electroporation). The methodology was applied to a model gene (Selectin P Ligand; Selplg) wherein there was 90% knockout of the gene in murine T cells 5-days after electroporation-based delivery of the Cas9 RNP complex (FIG. 2) containing two distinct crRNA species targeting the Selplg gene (named AA and AB).

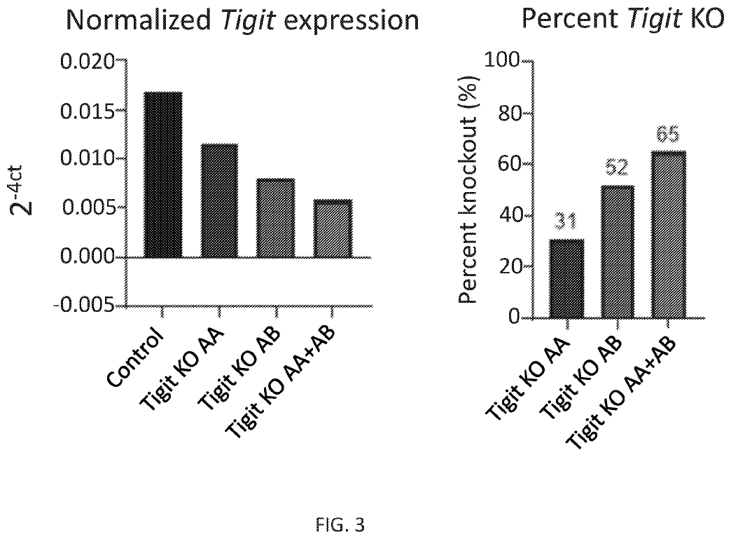

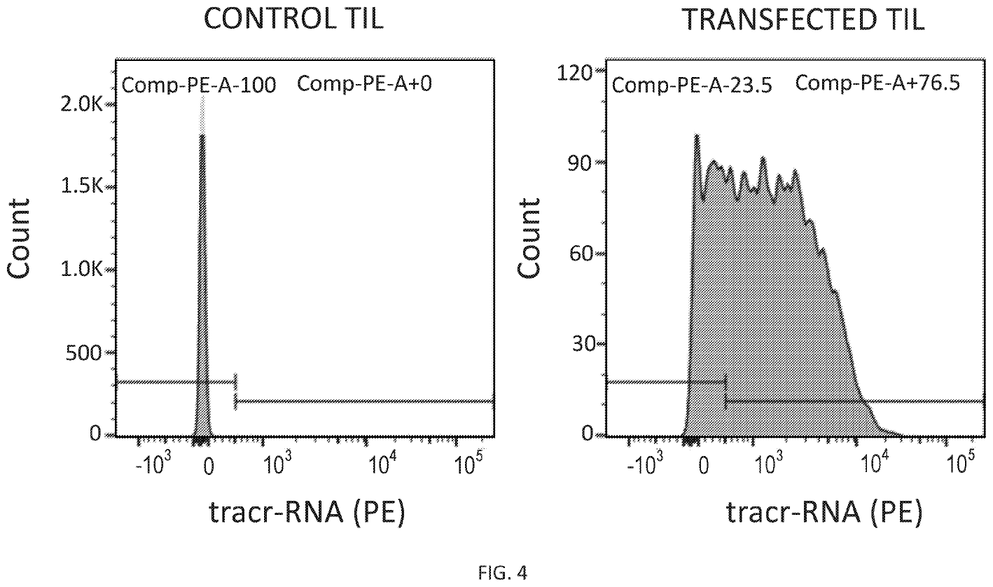

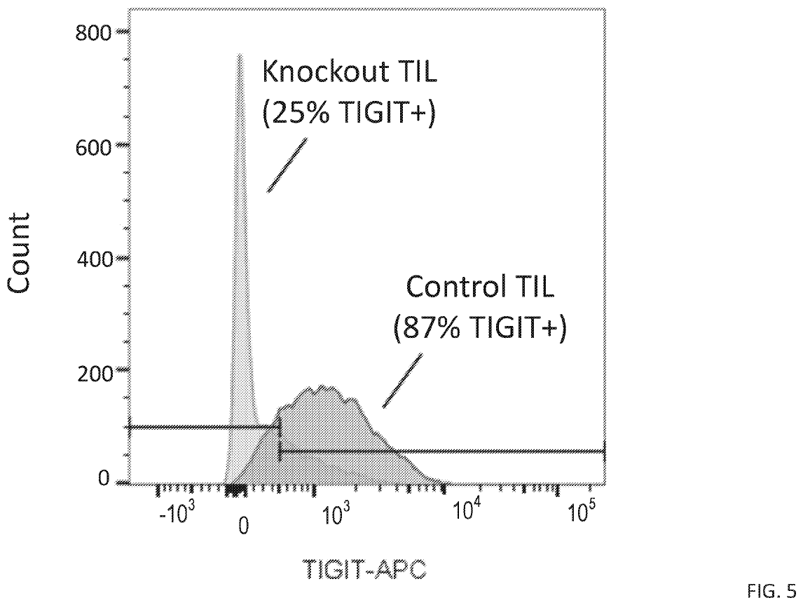

The methods were utilized in knocking out a gene of interest in murine T cells, as an example. FIG. 3 demonstrates the ability to knockout TIGIT in murine T cells using electroporation-based delivery of Cas9 RNP complexes. Data provided therein shows TIGIT knockout at the RNA level as assessed by RT-PCR. In FIG. 4, the inventors showed efficient engineering of TILs. Knockout of TIGIT is achieved with a transfection efficiency >75% in ex vivo-expanded patient-derived TIL. Transfer of TIGIT-specific RNP complexes by electroporation resulted in a cellular delivery efficiency of 76.5% (percent positive cells). Finally, transfecting previously-expanded TIL with Cas9 RNP complexes targeting TIGIT results in appreciable knockout. Shown here are the percentage of total live TIL positive for TIGIT in a control case (non-transfected) and an example case (transfected with TIGIT-specific RNP). In particular embodiments, the described methods are utilized for knocking out a different gene in TILs, such as TGF-beta R2, for example.

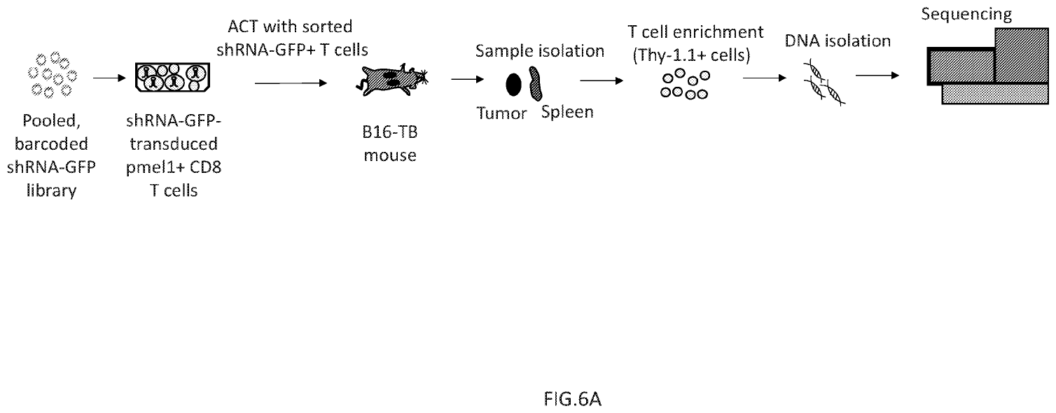

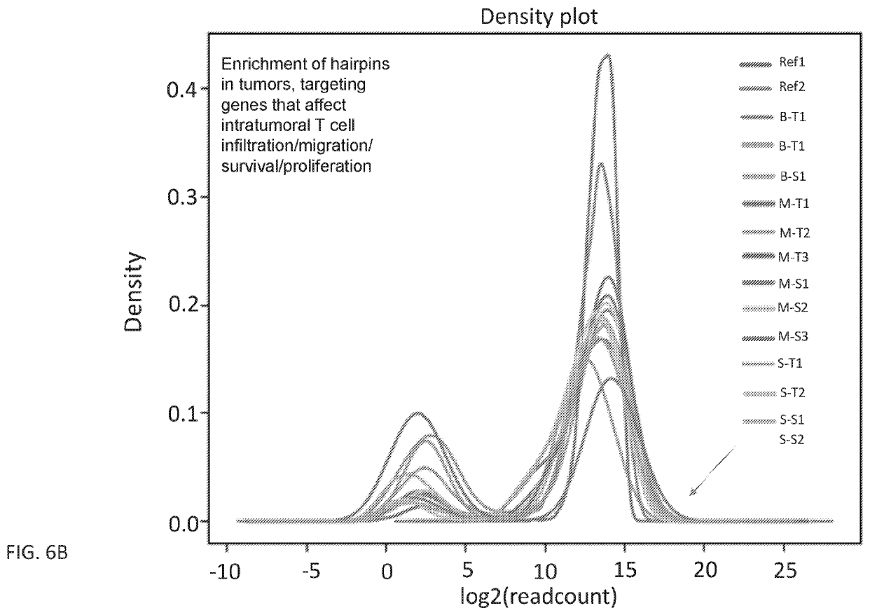

FIGS. 6A-6B show identification of genes that regulate T cell infiltration into tumors through pooled shRNA screens in vivo. 6A. Schematic representation of the experimental design. Activated pmel T cells were transduced with a pooled shRNA library targeting 300 genes encoding proteins expressed on the cell surface, and cells were adoptively transferred (ACT) into irradiated B16 tumor-bearing mice. 7 days post ACT, pmel T cells were isolated from B16 tumors and spleens paired samples, and DNA isolation and sequencing were performed. 6B. Density plot. The arrow in the density plot indicates the enriched hairpins in the TIL population compared to splenic T cells and references (samples acquired before ACT). Analysis was done for 2-3 samples per group. Representative surface T cell screen is shown.

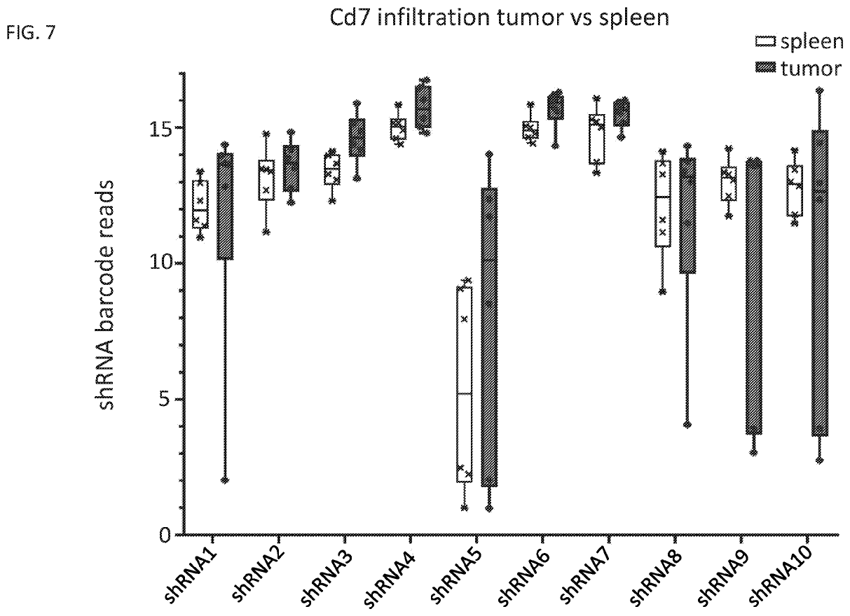

FIG. 7 shows enhancement of T cell infiltration in tumor versus spleen through Cd7 knockdown based on shRNA barcodes. The number of shRNA barcode reads for each of the 10 different shRNA constructs targeting Cd7 in both the spleen and tumor samples (n=6 each) is shown. The majority of constructs show enrichment in the tumor samples compared to the spleen samples.

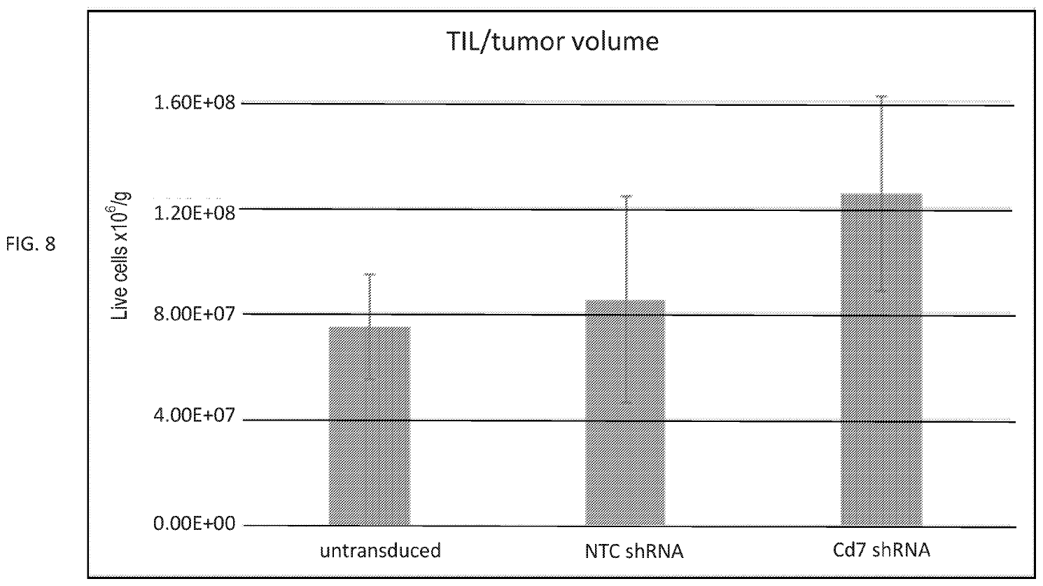

FIG. 8 shows enrichment of Cd7 knockdown Pmel in tumors versus spleens based on individual gene knockdown. Pmel T cells were separately transduced with either a Cd7 shRNA containing lentiviral vector or a non-targeting control (NTC) vector, FACS-sorted for vector-expressed GFP and expanded before ACT into tumor-bearing mice. Total tumor infiltrating immune lymphocytes (TILs) were isolated from the tumors 12 days after ACT and counted. The Cd7 knockdown Pmel were found in higher numbers compared with untransduced or NTC-construct transduced Pmel T cells, confirming the effect found in the shRNA screen.

Pmel T cells were separately transduced with either a Cd7 shRNA containing lentiviral vector or a non-targeting control (NTC) vector, FACS-sorted for vector-expressed mCherry and expanded before ACT into tumor-bearing mice. The day before ACT, Cd7 shRNA-transduced Pmel T cells expressed 93.9% mCherry and were 95.1% viable as analyzed by mCherry and live/dead flow cytometry analysis. qRT-PCR analysis confirmed Cd7 mRNA expression to be reduced by 84% as compared to NTC-transduced Pmel T cells.

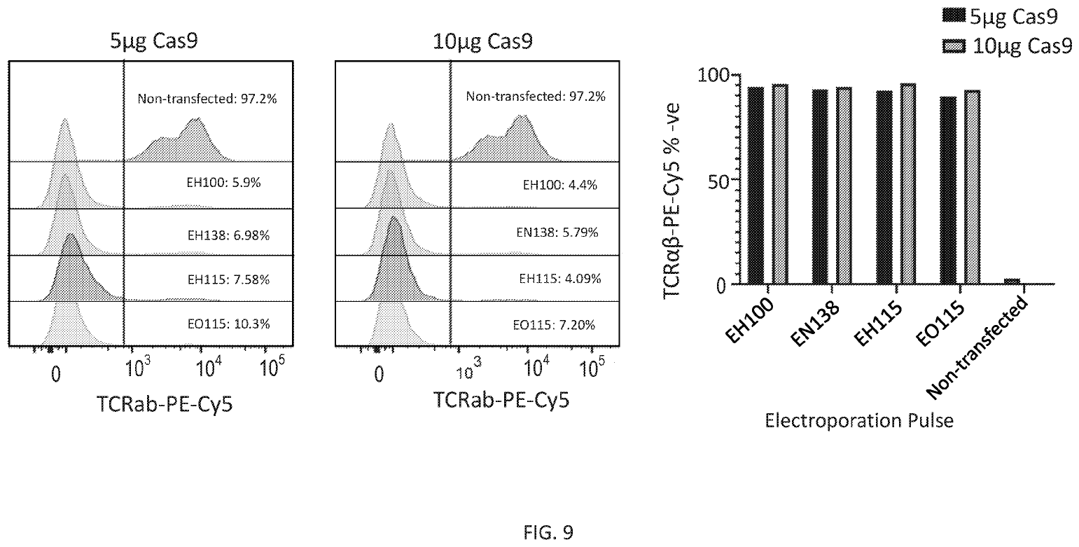

CRISPR gene knockout was optimized in patient-derived TIL using the T cell receptor alpha chain gene (TRAC) as a model target (FIG. 9). Across several different electroporation pulse parameters (denoted EH100, EN138, EH115, and E0115) and two different quantities of Cas9 input (5 g and 10 g), robust (>90%) elimination of the alpha beta T cell receptor was achieved. These data demonstrate robust CRISPR gene editing in human T cells using a clinically-relevant transfection protocol.

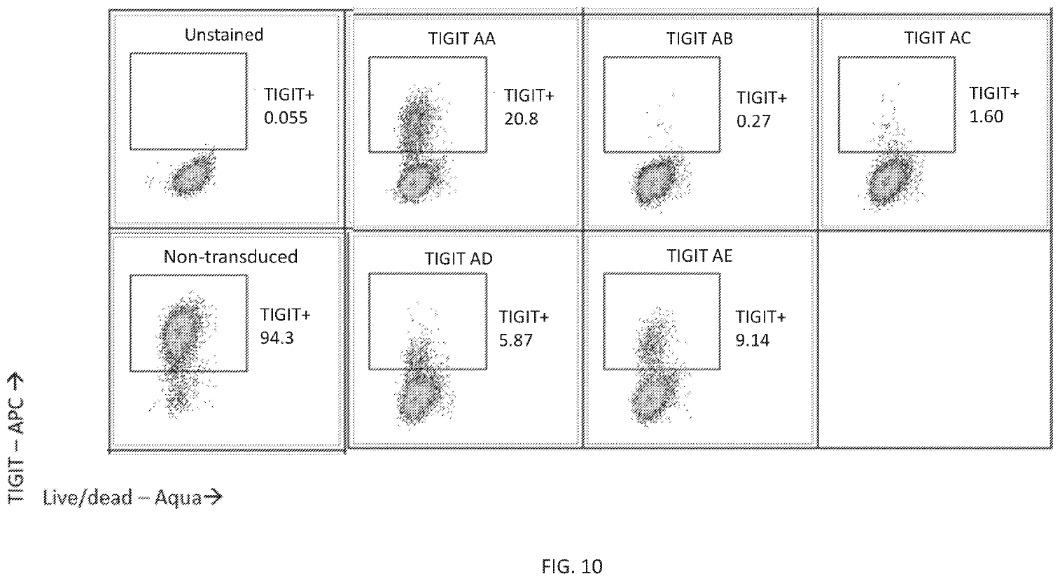

FIG. 10 concerns optimization of CRISPR gene knockout of TIGIT in patient-derived TIL. Un-modified TIL display 94.3% positivity for TIGIT surface expression. TIL subjected to TIGIT knockout with various guide RNA sequences (denoted TIGIT AA, AB, AC, AD, and AE) show reduced TIGIT surface expression. Use of the guide RNA sequences identified as “TIGIT AB” and “TIGIT AC” (merely as examples) show the strongest knockout efficiency with <2% of cells remaining positive for TIGIT surface expression after CRISPR gene editing.

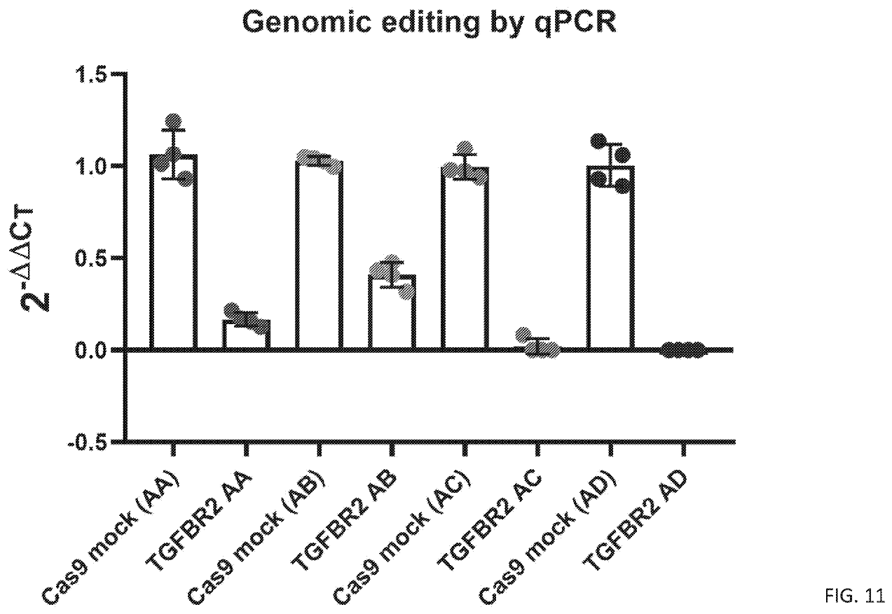

FIG. 11 demonstrates the optimization of CRISPR gene knockout of TGFBR2 in patient-derived TIL. TIL were genetically modified using guide RNAs targeting TGFBR2 (different guide RNA sequences denoted with TGFBR2 AA, AB, AC, and AD, as examples). Genetically modified TIL are characterized by substantially lower levels of the wild-type TGFBR2 sequence at the CRISPR cutsite compared to TIL transfected with a Cas9 mock. Genetic modification with the guide RNAs identified as “TGFBR2 AC” and “TGFBR2 AD” show the most robust elimination of wild-type DNA.

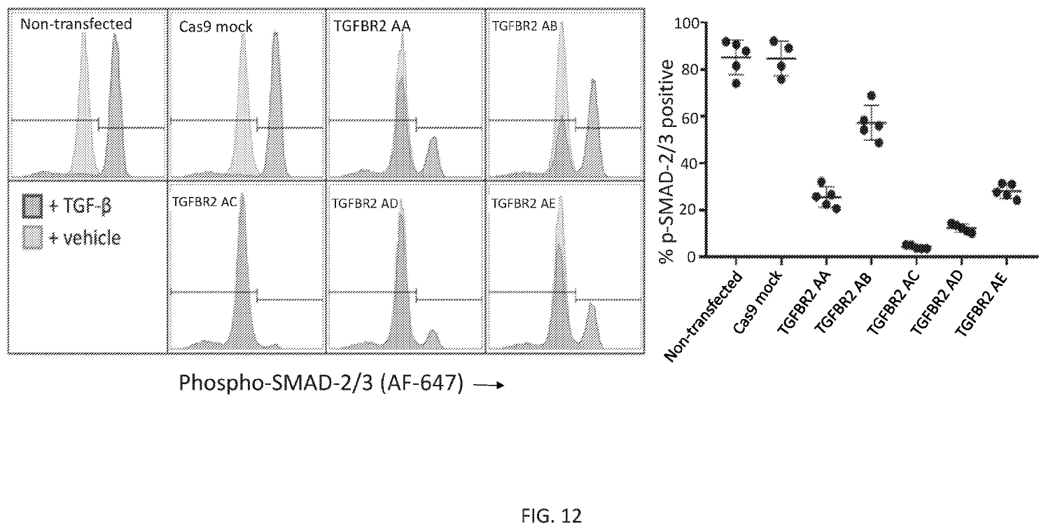

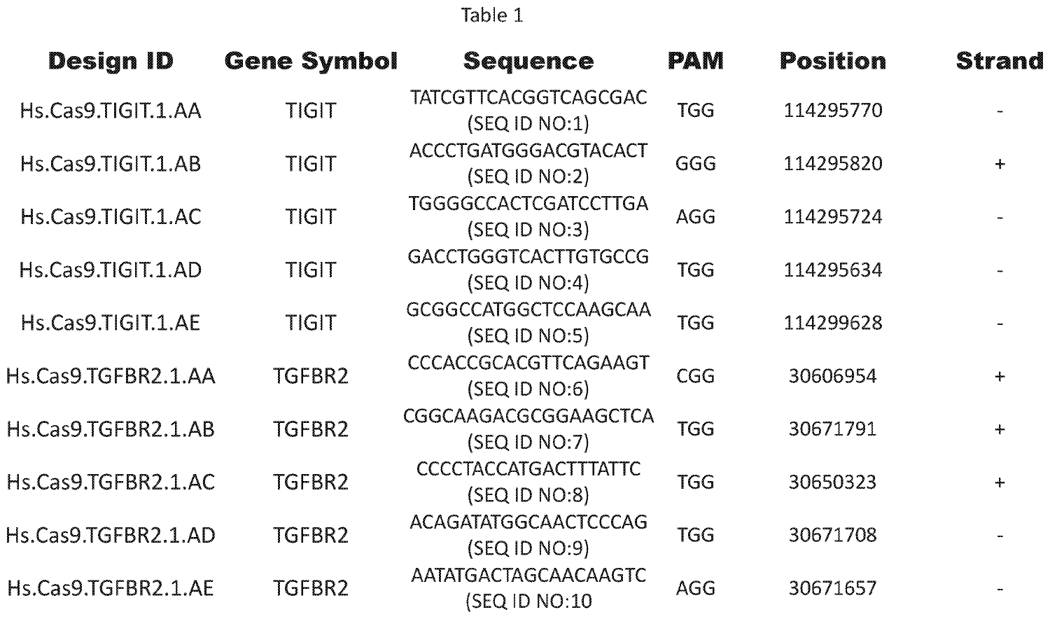

TIL that have undergone CRISPR gene knockout of TGFBR2 are resistant to the effects of exogenous TGF-β stimulation (FIG. 12). Un-modified TIL express high levels of phosphorylated SMAD-2 and SMAD-3 when exposed to exogenous TGF-β. In contrast, TIL that have undergone CRISPR gene editing to eliminate TGFBR2 are relatively resistant to TGF-β-induced SMAD phosphorylation. TIL were genetically modified using guide RNAs targeting TGFBR2 (different guide RNA sequences denoted with TGFBR2 AA, AB, AC, and AD, as examples). TIL modified with the guide RNAs named “TGFBR2 AC” and “TGFBR2 AD” demonstrated the strongest resistance to SMAD phosphorylation. These results were reproducible across several independent patient-derived TIL lines.

Table 1 in the figures provides examples of guide RNA sequences used to target TIGIT and TGFBR2 in human T cells. Guide RNAs were designed using the Integrated DNA Technologies (IDT) webtool.

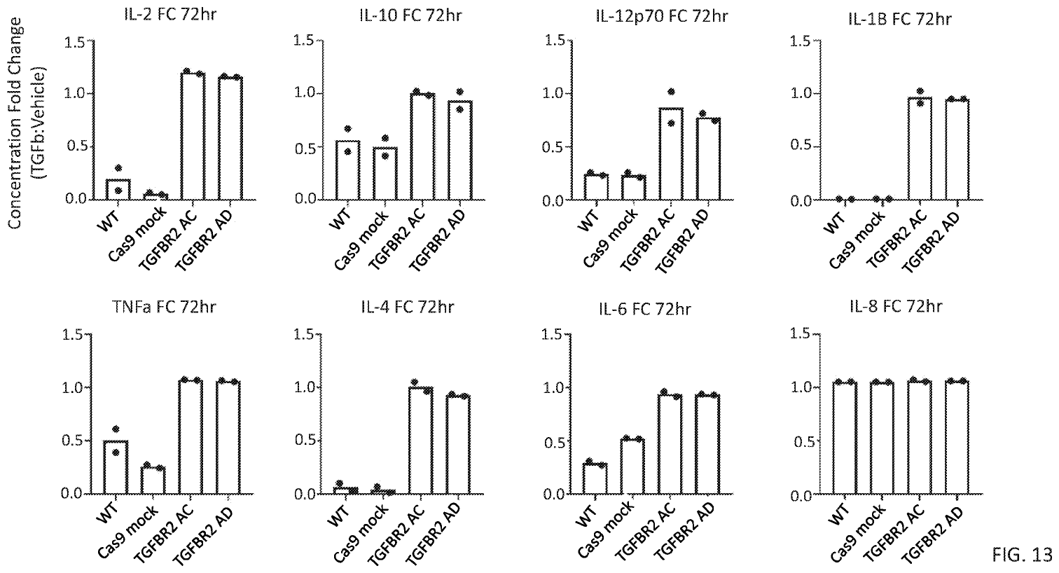

In FIG. 13, TIL that have undergone CRISPR gene knockout of TGFBR2 are resistant to the effects of exogenous TGF-β stimulation. Unmodified TIL secrete reduced levels of several pro-inflammatory cytokines when cultured in the presence of TGF-β for 3 days (as evidenced by a fold change in cytokine concentration from TGF-β:vehicle treated cells of <1.0). In contrast, TIL genetically modified using guide RNAs targeting TGFBR2 (different guide RNA sequences denoted with TGFBR2 AC and AD) secrete approximately equal amounts of pro-inflammatory cytokines when cultured in the presence or absence of TGF-β (as evidenced by a fold change TGF-β:vehicle of ˜1.0). Data are presented as the fold change (ratio) of cytokine concentration from TGF-β-treated (10 ng/ml) to vehicle treated TIL. Data are presented from TIL isolated from two independent donors.

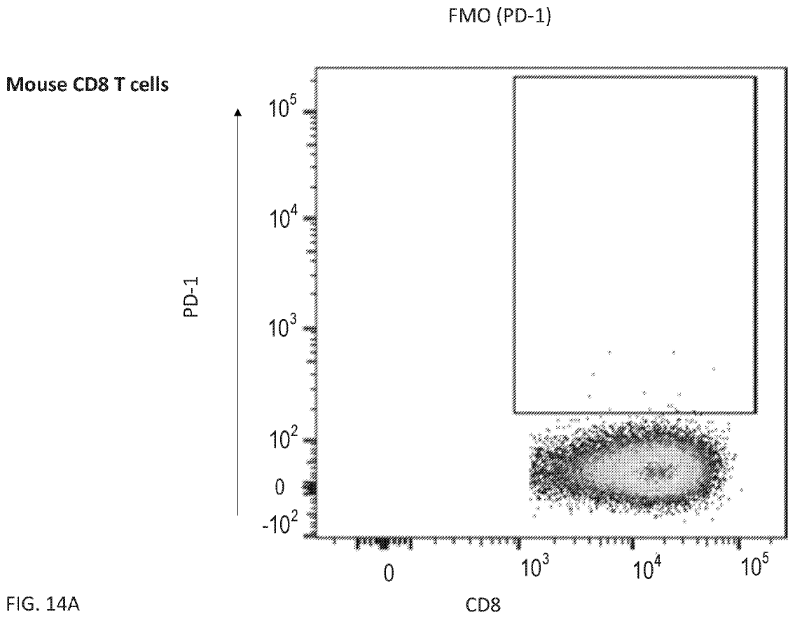

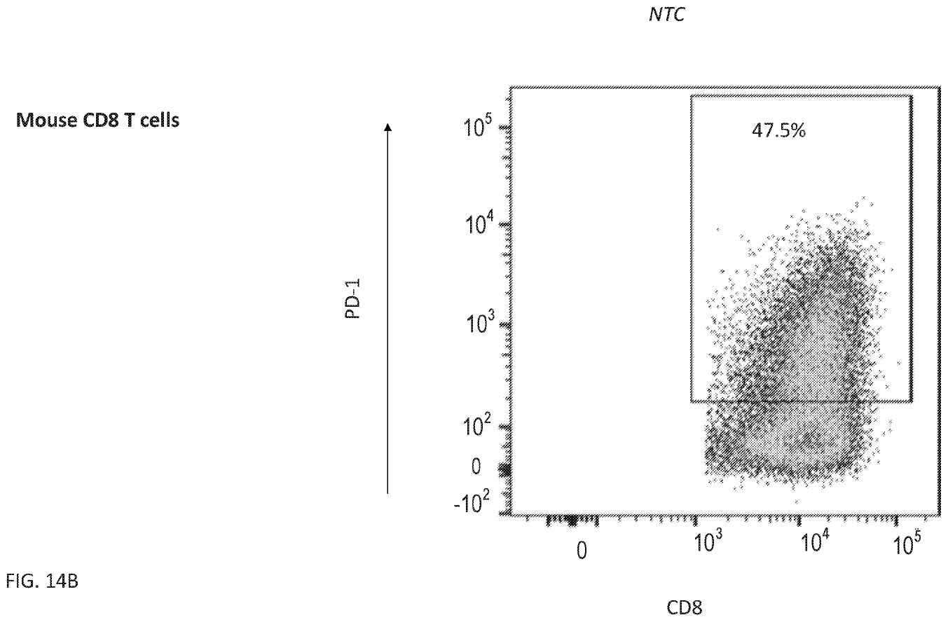

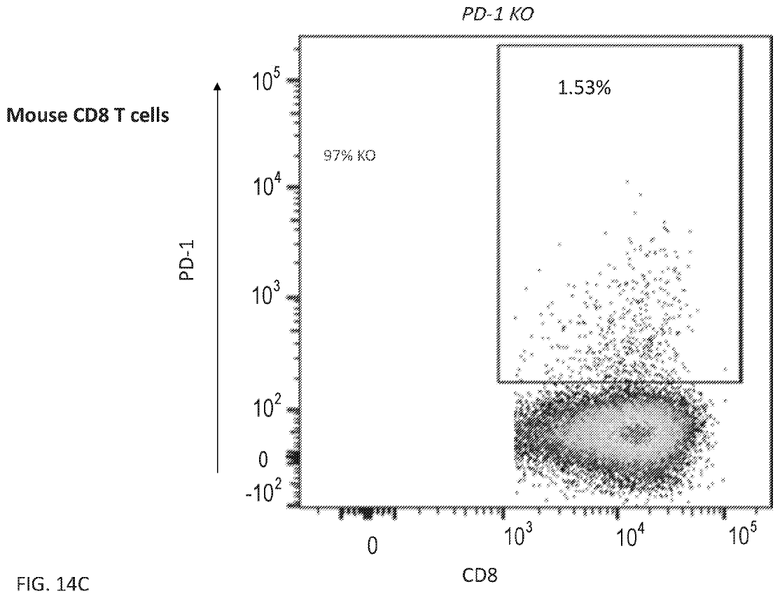

In FIG. 14, RNP transfection induces highly efficient Cas9/CRISPR-mediated PD-1 knockout in activated mouse CD8+ T cells. (14B) In vitro activation with anti-CD3 and IL-2 upregulates cell-surface expression of PD-1 in CD8 T cells (control cells transfected with non-targeting control RNP, NTC condition). (14C) T cells transfected with Cas9/gRNA RNP targeting PD-1, PD-1 KO condition, display a reduction of PD-1 expression, corresponding to 97% knockout efficiency compared to NTC expression. PD-1 protein expression evaluated by flow cytometry, six days after transfection; positive expression determined based on FMO (14A).

### REFERENCES

U.S. Pat. No. 5,399,363
U.S. Pat. No. 5,466,468
U.S. Pat. No. 5,543,158
U.S. Pat. No. 5,580,579
U.S. Pat. No. 5,629,001
U.S. Pat. No. 5,641,515
U.S. Pat. No. 5,725,871
U.S. Pat. No. 5,739,169
U.S. Pat. No. 5,756,353
U.S. Pat. No. 5,780,045
U.S. Pat. No. 5,792,451
U.S. Pat. No. 5,801,005
U.S. Pat. No. 5,804,212
U.S. Pat. No. 5,824,311
U.S. Pat. No. 5,830,880
U.S. Pat. No. 5,846,945
U.S. Pat. No. 6,613,308
Austin-Ward and Villaseca, Revista Medica de Chile, 126(7):838-845, 1998.
Bukowski et al., Clinical Cancer Res., 4(10):2337-2347, 1998.
Christodoulides et al., Microbiology, 144(Pt 11):3027-3037, 1998.
Davidson et al., J. Immunother., 21(5):389-398, 1998.
Hanibuchi et al., Int. J. Cancer, 78(4):480-485, 1998.
Hellstrand et al., Acta Oncologica, 37(4):347-353, 1998.
Hollander, Front. Immun., 3:3, 2012.
Hui and Hashimoto, Infection Immun., 66(11):5329-5336, 1998.
Mathiowitz et al., 1997.
Qin et al., Proc. Natl. Acad. Sci. USA, 95(24):14411-14416, 1998.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A composition, comprising:
(a) engineered tumor-infiltrating lymphocytes (TILs), wherein said TILs comprise one or more of (1) disruption of expression and/or activity of transforming growth factor-beta receptor 2 (TGFBR2); (2) disruption of expression and/or activity of T-cell-Ig-and-ITIM-domain (TIGIT); (3) disruption of expression and/or activity of CD7, all of which are endogenous to the TILs; (4) disruption of expression of programmed cell death protein 1 (PD-1); and (5) disruption of expression of T-cell immunoglobulin and mucin-domain containing-3 (TIM-3); and/or
(b) engineered T cells, wherein said T cells comprise one or more of (1) disruption of expression and/or activity of transforming growth factor-beta receptor 2 (TGFBR2) endogenous to the TILs; (2) disruption of expression and/or activity of T-cell-Ig-and-ITIM-domain (TIGIT); (3) disruption of expression and/or activity of CD7; (4) disruption of expression of PD-1; and (5) disruption of expression of TIM-3, all of which are endogenous to the T cells.

2. The composition of claim 1, wherein the TILs are expanded TILs and/or wherein the T cells are expanded T cells.

3. The composition of claim 1 or 2, wherein the disruption of expression and/or activity of one or more of TGFBR2, TIGIT, CD7, PD-1 and TIM-3 comprises nucleic acid, peptide, protein, small molecule, or a combination thereof.

4. The composition of claim 3, wherein the nucleic acid comprises siRNA, shRNA, anti-sense oligonucleotides, or guide RNA for CRISPR corresponding to TGFBR2, TIGIT, CD7, PD-1 and TIM-3, respectively.

5. The composition of any one of claims 1-4, wherein the TILs comprise disruption of expression of TGFBR2.

6. The composition of any one of claims 1-5, wherein the TILs comprise disruption of expression of TIGIT.

7. The composition of any one of claims 1-6, wherein the TILs comprise disruption of expression of CD7.

8. The composition of any one of claims 1-7, wherein the TILs comprise disruption of expression of PD-1.

9. The composition of any one of claims 1-8, wherein the TILs comprise disruption of expression of TIM-3.

10. The composition of any one of claims 1-9, wherein the T cells comprise disruption of expression of TGFBR2.

11. The composition of any one of claims 1-10, wherein the T cells comprise disruption of expression of TIGIT.

12. The composition of any one of claims 1-11, wherein the T cells comprise disruption of expression of CD7.

13. The composition of any one of claims 1-12, wherein the T cells comprise disruption of expression of PD-1.

14. The composition of any one of claims 1-13, wherein the T cells comprise disruption of expression of TIM-3.

15. The composition of any one of claim 1, 2, 3, 4, 10, 11, 12, 13, or 14, wherein the TILs or T cells comprise one or more heterologous antigen receptors that target one or more cancer antigens.

16. The composition of claim 15, wherein the heterologous antigen receptor is a T cell receptor, chimeric antigen receptor, chemokine receptor, chimeric cytokine receptor, or a mixture thereof.

17. A population of cells of the composition of any one of claims 1-16.

18. A composition, comprising the population of claim 17.

19. The composition of claim 18, wherein the population is in a pharmaceutically acceptable carrier.

20. A method of preparing the cells of any one of claims 1-16, comprising the step of electroporating the TILs and/or T cells, respectively with:
(a) Cas9 or a nucleic acid that encodes Cas9; and one or more of (b), (c), (d), (e), and (f):
(b) one or more TGFBR2 guide RNAs for CRISPR;
(c) one or more TIGIT guide RNAs for CRISPR; or
(d) one or more CD7 guide RNAs for CRISPR;
(e) one or more PD-1 guide RNAs for CRISPR; and
(f) one or more TIM-3 guide RNAs for CRISPR.

21. The method of claim 18, further defined as comprising two or more electroporation steps, wherein a first electroporation step subjects the TILs and/or engineered T cells to one or more of TGFBR2 guide RNA, TIGIT guide RNA, CD7 guide RNA, PD-1 guide RNA, and TIM-3 guide RNA, and a second electroporation step subjects the TILs and/or engineered T cells to guide RNAs for one or more of TGFBR2, TIGIT, CD7, PD-1, and TIM-3 that were not used in the first electroporation step.

22. The method of any one of claims 20-21, further comprising at least one step of expanding the TILs and/or T cells.

23. The method of claim 22, wherein there is an expansion step for the TILs and/or T cells prior to an electroporation step.

24. The method of claim 22 or 23, wherein there is an expansion step for the TILs and/or T cells after to an electroporation step.

25. The method of any one of claims 20-24, further comprising the step of modifying the T cells or TILs to express one or more heterologous antigen receptors.

26. The method of claim 25, wherein the heterologous antigen receptor is a T cell receptor, chimeric antigen receptor, chemokine receptor, chimeric cytokine receptor, or a mixture thereof.

27. The method of claim 25 or 26, wherein the heterologous antigen receptor is customized to target a cancer antigen on cancer cells of an individual.

28. A method of killing cancer cells in an individual, comprising the step of delivering to the individual a therapeutically effective amount of the composition of any one of claims 1-16.

29. The method of claim 28, wherein the cancer is a hematological cancer or comprises a solid tumor.

30. The method of any one of claims 28-29, wherein the TILs and/or T cells are allogeneic with respect to the individual.

31. The method of any one of claims 28-29, wherein the TILs and/or T cells are autologous with respect to the individual.

32. The method of any one of claims 28, 29, or 31, further defined as:
(a) obtaining cancer cells from the individual;
(b) expanding TILs from the cancer cells to produce expanded TILs;
(c) engineering the expanded TILs to have (1) disruption of expression or activity of TGFBR2 endogenous to the TILs; and/or (2) disruption of expression or activity of TIGIT endogenous to the TILs; and/or (3) disruption of expression or activity of CD7 endogenous to the TILs; and/or (4) disruption of expression or activity of PD-1 endogenous to the TILs; and/or (5) disruption of expression or activity of TIM-3 endogenous to the TILs; and
(d) administering an effective amount of the engineered cells to the individual.

33. The method of any one of claims 28-32, wherein the individual is delivered an additional cancer therapy.

34. The method of claim 33, wherein the additional cancer therapy comprises surgery, radiation, chemotherapy, hormone therapy, immunotherapy, or a combination thereof.
